(19) United States
(12) Patent Application Publication
(10) Pub. No.: US 2005/0113791 A1
(43) Pub. Date: May 26, 2005

(54) Zoned absorbent structures and process for producing same

(75) Inventors: Andrew E. Neubauer, Menasha, WI (US); Gary N. Attoe, Hortonville, WI (US); Steven P. Wolf, Sherwood, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/721,834

(22) Filed: Nov. 24, 2003

(51) Int. Cl. A61F 13/15; A61F 13/20

(52) U.S. Cl. 604/387

(57) ABSTRACT

A system and process for forming absorbent structures is disclosed. Also disclosed are absorbent structures suitable for incorporation into absorbent products, such as diapers, a training pant, adult incontinence products, feminine hygiene products, and the like. The absorbent structures include particularly shaped openings and a pair of opposing lateral flaps. When the lateral flaps are folded, the lateral flaps in conjunction with the openings form an absorbent structure having a high basis weight area in a desired location. The absorbent structures can be formed from a web having a uniform basis weight and may be formed without having to scarf the absorbent web as was required in many prior processes. In order to form the openings, masking members may be attached to a forming surface in an air forming process. The masking members are convertable for forming gender specific products if desired.

### BACKGROUND OF THE INVENTION

Many types of disposable consumer products such as diapers, training pants, feminine care articles, incontinence articles, and the like, utilize an absorbent pad structure for absorbing and wicking away bodily fluids. The absorbent structures are conventionally formed from an absorbent pad or batt, typically a fibrous material. With one particular general practice, the absorbent web is formed by employing conventional airlaying techniques wherein fibers and typically a superabsorbent material are mixed and entrained in an air stream and then directed onto a forming surface to form the web. The absorbent web may then be directed for further processing and assembly with other components to produce a final absorbent article. An advantage of this practice is that trim waste that may be removed from the absorbent structure can be immediately recycled by returning the waste to the upstream fiberizing equipment and/or airlaying equipment.

With another conventional technique, preformed absorbent web sheets or layers are delivered into a manufacturing line from a preformed supply, such as a supply roll. The absorbent sheet material may be separated into adjacent strips having various configurations of repeat pattern, and/or “nested” shaped pads wherein the shape of one pad is substantially nested with the shape of at least one immediately adjacent pad.

The preformed absorbent material roll process presents particular challenges. For example, the geographical separation of the base roll-making machine makes recycling of the trim waste impractical and cost prohibitive. In this regard, the nesting feature mentioned above has been desirable to reduce the amount of waste that is generated from the originally supplied (roll) of absorbent web. However, with conventional nesting techniques and profiles, a considerable amount of trim waste can still be generated.

Also, the more easily processed strip-shapes have a repeat pattern that is substantially symmetrical with respect to its longitudinal dimension that coincides with the machine direction of the web. With such longitudinally-symmetric nested patterns, a single cycle of the repeat pattern provides an individual web segment wherein the shape of a first lengthwise half portion of the segment substantially matches the shape of the longitudinally opposed other half portion. However, for certain consumer absorbent articles, it has been found desirable from a product fit, comfort, and performance standpoint to shape the pad so that it is longitudinally asymmetric. For example, the pad may have a wider front or “ear” portion as compared to the back portion. Unfortunately, such configurations in a nested pattern add to the amount of generated waste.

Also, it may be desirable to provide a higher basis weight of absorbent material in the crotch portion as compared to the front and back portions. This has been conventionally done by using a forming surface in an air forming process that contains pockets. The pockets have a depth greater than other portions of the forming surface. Thus, during the air forming process, fibers and absorbent particles collect in the pockets creating greater basis weight areas.

Unfortunately, in certain configurations, the pockets cannot be filled completely without overfilling the non-pocket regions. Consequently, the formed fibrous web has to be scarfed in order to remove absorbent material in the non-pocket regions. Scarfing is a process in which a rotating brush or other suitable device contacts the fibrous material in order to remove unwanted portions. Scarfing, for instance, is described in U.S. Pat. No. 6,416,697, which is incorporated herein by reference. The scarfed fibrous material is then returned to the forming chamber and reused.

In addition to having to scarf the final product, use of a pocketed forming surface has also other limitations. For instance, basis weight ratios are limited by the process. Further, scarfing cannot practically be performed when various components are contained in the fibrous material that is used to form the absorbent layer. For instance, scarfing is not well suited for absorbent structures with very high superabsorbent material/fluff ratios or absorbents with components such as meltblown fibers, which may be added in certain situations to improve integrity.

In addition to creating high basis weight areas in absorbent structures, it is also desirable in many applications to change the location of the high basis weight areas depending upon the particular product being made. For instance, it may be desirable to change the location of the high basis weight area in gender specific products. For example, in products made for girls or for women, it is generally desirable to have a high basis weight area in the crotch area of the garment. In products designed for boys and men, on the other hand, it may be desirable for the high basis weight area to extend from the crotch to the front portion of the product.

One problem involved in the production of gender specific products is in being able to configure a process line that can produce not only absorbent structures intended for male products but also produce absorbent structures intended for female products. In particular, a need currently exists for a process line that can be quickly switched between the production of female specific products and male specific products without having to completely change or reconfigure the forming surface, which may require excessive machine down time.

The present invention provides a method for producing longitudinally symmetric or asymmetric absorbent pad structures in a drum-forming process with minimal or zero waste of the absorbent material. The present invention also provides a method and process of producing absorbent structures having high basis weight areas. Further, through the present invention, the location of the high basis weight areas may be moved and/or altered for creating gender specific products.

### SUMMARY OF THE INVENTION

Various features and advantages of the invention will be set forth in part in the following description, or may be obvious from the description, or may be learned through practice of the invention.

The present invention provides an improved method for making absorbent structures for use in various applications of consumer disposable absorbent articles, such as disposable diapers, child's training pants, feminine care articles including but not limited to interlabial products, incontinence articles, swim pants, and the like. The structures may be longitudinally symmetric or asymmetric.

In one embodiment of the present invention, for instance, an absorbent article is provided comprising an outer cover material, a liner, and an absorbent structure positioned between the outer cover material and the liner. In accordance with the present invention, the absorbent structure comprises a front portion, a middle portion, a rear portion, a pair of opposing lateral flaps, a pair of opposing middle openings, and at least one rear opening. The pair of opposing middle openings are spaced between the middle portion and the lateral flaps. The lateral flaps are folded onto at least the middle portion. The middle portion has a width narrower than the width of the front portion due to the location of the pair of opposing middle openings. The folded lateral flaps create a basis weight in the location of the middle portion that may be at least about twice the basis weight of areas of the rear portion.

When the absorbent structure is for producing absorbent articles designed for males, the folded lateral flaps may also create a basis weight in the location of the front portion that may be at least about twice the basis weight of areas of the rear portion.

By including middle openings and the at least one rear opening and by including lateral flaps that fold inwardly, the absorbent structure of the present invention may be configured to have higher basis weight areas where desired. Further, the absorbent structures can be made with little or no waste. Further, by changing the size and/or the location of the middle openings and the at least one rear opening, gender specific absorbent structures may be formed.

In one embodiment, the middle openings may have an inner concave-shaped edge and an outer convex-shaped edge that cooperate when the lateral flaps are folded to give the absorbent structure an overall hourglass-like shape. The absorbent structure may include two opposing rear openings separated by a strip of material. The strip of material is connected to the middle portion and is narrower than the middle portion. The strip of material is included in the absorbent structure in order to provide integrity, especially during formation of the absorbent structure. In many applications, the one or more rear openings are connected to the corresponding middle openings.

The lateral flaps, once folded, may be adhesively secured to the middle portion. In one embodiment, a blank that is used to form the absorbent structure has an overall rectangular shape. In this embodiment, the lateral flaps may extend substantially the entire length of the absorbent structure. Consequently, the lateral flaps fold onto the front portion, the middle portion and the rear portion.

The absorbent structure can be made from any suitable liquid absorbent material. For example, in one embodiment, the absorbent structure may comprise pulp fibers and superabsorbent particles. The absorbent structure may be airformed, coformed, or made in any other suitable manner. The absorbent structure may have a basis weight of from about 100 grams per square meter (gsm) to about 2000 gsm and may have a density of from about 0.1 grams per cubic centimeter (g/cc) to about 0.45 g/cc.

In accordance with one embodiment of the present invention for making absorbent structures, an absorbent web material is formed and supplied in a machine-direction flow in the form of a continuous strip. The strip may include a succession of interconnected individual absorbent pads. Each of the pads may include a front portion, a middle portion, a rear portion, a pair of opposing lateral flaps, a pair of opposing middle openings, and at least one rear opening.

As the absorbent web material is conveyed in the machine direction, the opposing lateral flaps are folded onto at least the middle portion of each individual absorbent pad. The strip of web material is then cut in a cross direction into the individual absorbent pads. Once folded and cut, the middle portion of each pad may have a width narrower than the width of the front portion due to the location of the pair of opposing middle openings. Once folded, the lateral flaps create a basis weight in the location of the middle portion that is at least about twice the basis weight of areas of the rear portion. Similarly, the lateral flaps may also create a basis weight in the location of the front portion that is also at least about twice the basis weight of areas of the rear portion.

The pair of opposing middle openings and the at least one rear opening may be formed by cutting the absorbent web material. Alternatively, the opposing middle openings and the at least one rear opening may be formed during an air forming process in which the strip of web material is formed.

In order to assist in folding the lateral flaps, in one embodiment, the absorbent web material may be scored to form a pair of score lines that generally extend in the machine direction. The score lines define the lateral flaps. The absorbent web material may also be debulked during the process. For example, the absorbent web material may be debulked during formation of the score lines.

The inventions will be described below in greater detail by reference to particular embodiments set forth in the figures.

### BRIEF DESCRIPTION OF THE FIGURES

FIG. 1 is a perspective view of a representative absorbent article incorporating an absorbent structure in accordance with the present invention;

FIG. 2 is a perspective view of another embodiment of a representative absorbent article incorporating an absorbent structure in accordance with the present invention;

FIG. 3 is a plan view of the absorbent article as shown in FIG. 2 in an unfolded state;

FIG. 4 is another plan view of an absorbent article;

FIG. 5 is a perspective view of one embodiment of an absorbent structure made in accordance with the present invention;

FIG. 6 is a plan view of the absorbent structure shown in FIG. 5;

FIG. 7 is a plan view of a blank that may be used to form the absorbent structure illustrated in FIG. 5;

FIG. 8 is a plan view of a strip of material showing a succession of blanks as shown in FIG. 7;

FIG. 9 is a perspective view of another embodiment of an absorbent structure made in accordance with the present invention;

FIG. 10 is a plan view of a blank that may be used in forming the absorbent structure illustrated in FIG. 9;

FIG. 11 is a plan view of a strip of web material showing a succession of the blanks illustrated in FIG. 10;

FIG. 12 is a perspective view of a forming surface that may be used in making absorbent structures in accordance with the present invention;

FIG. 13 is a plan view of a forming surface showing masking members that may be used to make absorbent structures in accordance with the present invention;

FIG. 14 is a plan view of an alternative embodiment of a forming surface showing masking members for use in making absorbent structures in accordance with the present invention;

FIG. 15 is a perspective view of one embodiment of a process for forming absorbent structures in accordance with the present invention;

FIG. 16 is a perspective view of one embodiment of a process for forming absorbent articles incorporating the absorbent structures of the present invention;

FIG. 17 is a plan view of another embodiment of a blank that may be used to form absorbent structures in accordance with the present invention;

FIG. 18 is a plan view of an absorbent structure made in accordance with the present invention from the blank shown in FIG. 17; and

FIG. 19 is a perspective view of a forming surface that may be used to form a blank as shown in FIG. 17.

### DETAILED DESCRIPTION

The invention will now be described in detail with reference to particular embodiments thereof. The embodiments are provided by way of explanation of the invention, and not meant as a limitation of the invention. For example, features described or illustrated as part of one embodiment may be used with another embodiment to yield still a further embodiment. It is intended that the present invention include these and other modifications and variations as come within the scope and spirit of the invention.

The present method is particularly suited for the manufacture of pad structures from a web of absorbent material. The pads are intended for use in various consumer disposable absorbent products. Such products include, but are not limited to, diapers, children's training pants, feminine care articles (such as panty liners, pads, and interlabial products), incontinence articles, swim pants, and the like. The invention is not limited to any particular type or composition of absorbent web material, and may be practiced with any suitable absorbent web material known to those skilled in the art. The absorbent web material may include any structure and combination of components which are generally compressible, conformable, non-irritating to a wearer's skin, and capable of absorbing and retaining liquids and certain body wastes.

The absorbent pad structures of the present invention include higher basis weight areas in desired locations. For instance, the higher basis weight areas may be formed into the crotch area of an absorbent article. The higher basis weight areas may also extend from the crotch area into a front portion of the absorbent article. In accordance with the present invention, the absorbent pads contain openings and lateral flaps that are folded during formation of the absorbent structures. Through the use of openings having particular shapes and through the use of lateral flaps, the higher basis weight areas may be formed into the absorbent structures without, in one embodiment, creating any waste. In accordance with the present invention, the shape and location of the openings may be varied for creating gender specific products.

The material used to form the absorbent structures, for example, may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from US Alliance Pulp Mills of Coosa, Ala., USA, and is a bleached, highly absorbent wood pulp containing primarily soft wood fibers. As a general rule, the superabsorbent material is present in the absorbent web in an amount of from about 0 to about 90 weight percent based on total weight of the web. The web may have a density within the range of about 0.1 to about 0.45 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a suberabsorbent material is capable of absorbing at least about 15 times its weight in liquid, and suitably is capable of absorbing more than about 25 times its weight in liquid. Suitable superabsorbent materials are readily available from various suppliers. For example, FAVOR SXM 880 superabsorbent is available from Stockhausen, Inc., of Greensboro, N.C., USA; and Drytech 2035 is available from Dow Chemical Company, of Midland, Mich., USA.

In addition to cellulosic fibers and superabsorbent materials, the absorbent pad structures may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives can enhance the integrity of the bulk absorbent structure, and alternatively or additionally may provide adherence between facing layers of the folded structure.

Subsequent to or after being cut from the web material strip, the individual absorbent pads may be partially or wholly wrapped or encompassed by a suitable tissue or nonwoven wrap that aids in maintaining the integrity and shape of the pad.

The absorbent materials may be formed into a web structure by employing various conventional methods and techniques. For example, the absorbent web may be formed with a dry-forming technique, an airlaying technique, a carding technique, a meltblown or spunbond technique, a wet-forming technique, a foam-forming technique, or the like, as well as combinations thereof. Layered and/or laminated structures may also be suitable. Methods and apparatus for carrying out such techniques are well known in the art.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles or fibers, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. Nos. 4,100,324 to Anderson, et al.; U.S. Pat. No. 5,284,703 to Everhart, et al.; and U.S. Pat. No. 5,350,624 to Georger. et al.; which are incorporated herein in their entirety by reference for all purposes.

It is also contemplated that elastomeric absorbent web structures may be particularly well suited to the present invention. For example, an elastomeric coform absorbent structure having from about 35% to about 65% by weight of a wettable staple fiber, and greater than about 35% to about 65% by weight of an elastomeric thermoplastic fiber may be used to define absorbent pad structures according to the invention. Examples of such elastomeric coform materials are provided in U.S. Pat. No. 5,645,542, incorporated herein in its entirety for all purposes. As another example, a suitable absorbent elastic nonwoven material may include a matrix of thermoplastic elastomeric nonwoven filaments present in an amount of about 3 to less than about 20% by weight of the material, with the matrix including a plurality of absorbent fibers and a super-absorbent material each constituting about 20-77% by weight of the material. U.S. Pat. No. 6,362,389 describes such a nonwoven material and is incorporated herein by reference in its entirety for all purposes. Absorbent elastic nonwoven materials are useful in a wide variety of personal care articles where softness and conformability, as well as absorbency and elasticity, are important.

The absorbent web may also be a nonwoven web comprising synthetic fibers. The web may include additional natural fibers and/or superabsorbent material. The web may have a density in the range of about 0.1 to about 0.45 grams per cubic centimeter. The absorbent web can alternatively be a foam.

In a particular aspect of the invention, the absorbent web material can be provided with an absorbent capacity of at least about 8 g/g employing 0.9 wt % saline (8 grams of 0.9 wt % saline per gram of absorbent web). The absorbent capacity of the absorbent web can alternatively be at least about 9 g/g, and can optionally be at least about 15 g/g to provide improved benefits. Additionally, the absorbent capacity may be up to about 40 g/g, or more, to provide desired performance.

In another aspect, the web of absorbent material can be provided with a tensile strength value of at least about 0.5 N/cm (Newtons per cm of “width” of the material, where the “width” direction is perpendicular to the applied force). The tensile strength of the absorbent web can alternatively be at least about 1.5 N/cm, and can optionally be at least about 2 N/cm to provide improved benefits. In another aspect, the web of absorbent material can be provided with a tensile strength value of up to a maximum of about 100 N/cm, or more. The tensile strength of the absorbent web can alternatively be up to about 10 N/cm, and can optionally be up to about 20 N/cm to provide improved benefits.

The selected tensile strength should provide adequate processibility of the web throughout the manufacturing process, and can help to produce articles that exhibit desired combinations of softness and flexibility. In particular, the absorbent web material should have a tensile strength in the cross-direction to undergo stretching as described herein without resulting in substantial degradation of the web integrity to the extent that the pad structures cannot be further processed in absorbent articles. In some cases, the stretching of the web material in the cross direction can provide a softer and more flexible material than the initial web. This is generally desired for initially stiff materials such as some stabilized airlaid or wetlaid materials.

The absorbent material web is also selected so that the individual absorbent pad structures possess a particular individual total absorbency depending on the intended article of use. For example, for infant care products, the total absorbency can be within the range of about 200-900 grams of 0.9 wt % saline, and can typically be about 500 g of 0.9 wt % saline. For adult care products, the total absorbency can be within the range of about 400-2000 grams of 0.9 wt % saline, and can typically be about 1300 g of saline. For feminine care products, the total absorbency can be within the range of about 7-50 grams of menstrual fluid or menses simulant, and can typically be within the range of about 30-40 g of menstrual fluid or menses simulant.

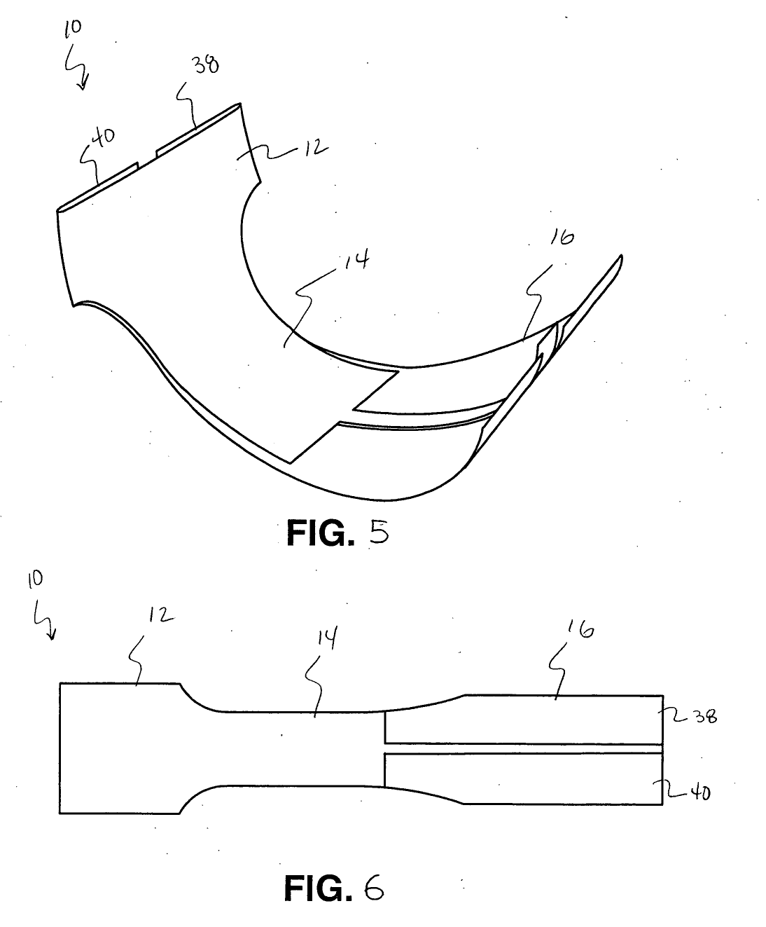

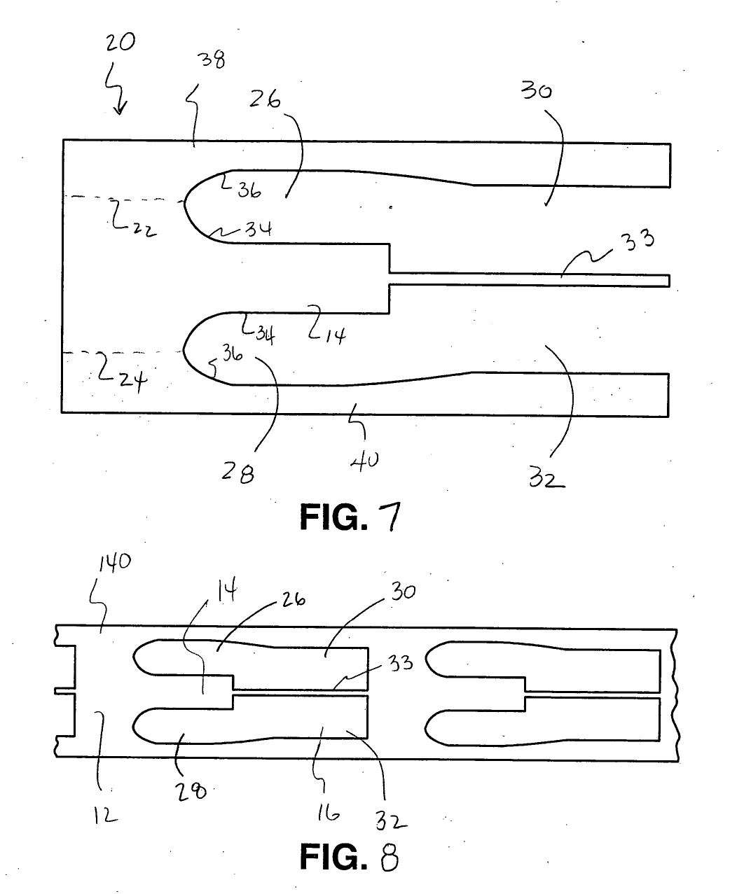

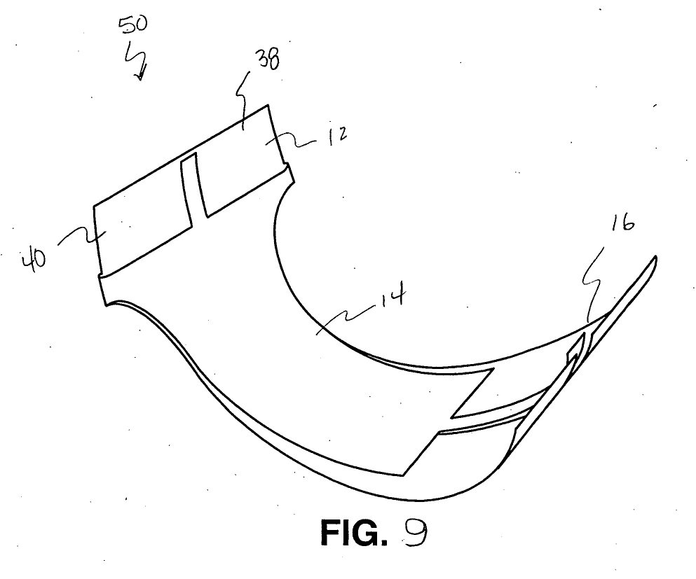

Referring now to FIGS. 5 and 9, two embodiments of absorbent structures made in accordance with the present invention are illustrated. For instance, referring to FIG. 5, an absorbent structure generally 10 is shown. A top plan view of the absorbent structure 10 is also shown in FIG. 6. The absorbent structure 10 includes a front portion 12, a middle portion 14, and a rear portion 16. When incorporated into an absorbent product, the middle portion 14 is positioned generally in the crotch area of the garment, while the front portion 12 is positioned adjacent to the front of a wearer.

Referring to FIG. 7, a blank generally 20 is shown that may be used to form the absorbent structure 10 as shown in FIGS. 5 and 6. As shown, the blank 20 has a generally rectangular shape and includes score lines 22 and 24. The blank 20 includes a pair of opposing middle openings 26 and 28 and a pair of opposing rear openings 30 and 32. As shown, the middle portion 14 is positioned in between the middle openings 26 and 28. In this embodiment, the rear openings 30 and 32 are interconnected and continuous with the middle openings 26 and 28. In other embodiments, however, the rear openings 30 and 32 may be separate from the middle openings 26 and 28. Further, in other embodiments, only a single rear opening may be needed.

The rear openings 30 and 32 of the blank 20 shown in FIG. 7 are also separated by a thin strip of material 33. The thin strip of material 33 is for providing integrity to the blank 20 when part of a continuous roll or strip of material.

Each middle opening 26 and 28 includes an arcuate-shaped edge 34 and a corresponding arcuated-shaped edge 36, which extends in an opposite direction. As shown, the score lines 22 and 24 separate the arcuate-shaped edges.

The score lines 22 and 24 also define a pair of opposing lateral flaps 38 and 40. In order to convert the blank 20 as shown in FIG. 7 into the absorbent structure 10 as shown in FIGS. 5 and 6, the lateral flaps 38 and 40 are folded over onto the front portion 12, the middle portion 14, and the rear portion 16. As shown in FIGS. 5 and 6, when the lateral flaps 38 and 40 are folded, the middle openings 26 and 28 provide the absorbent structure 10 with an overall hourglass-like shape. Further, the folded lateral flaps in conjunction with the rear openings create basis weight differentials over the length of the absorbent structure 10.

For example, as shown in FIG. 5, once the lateral flaps 38 and 40 are folded, the front portion 12 and the middle portion 14 comprise two layers of material, while the rear portion 16 comprises primarily only a single layer of material. Thus, when the absorbent is formed to have a substantially uniform basis weight at all locations before folding, the front portion 12 and the middle portion 14 can have a basis weight that is at least about twice the basis weight of the rear portion 16 after folding. In other embodiments, however, the blank 20 may be formed so as to have basis weight differentials. For example, the lateral flaps may have a basis weight greater than the basis weight of the front portion, the middle portion or the rear portion. In other embodiments, for instance, the middle portion may have a basis weight greater than the lateral flaps, the front portion or the rear portion. Generally, once the lateral flaps 38 and 40 are folded, the front portion 12 and the middle portion 14 may have a basis weight, for instance, that is at least 25% greater than the basis weight of the rear portion 16, particularly at least 50% greater, and more particularly at least 100% greater.

In the embodiment shown in FIG. 5, the lateral flaps 38 and 40 have a width that is approximately one half the width of the middle portion 14. In other embodiments, however, the width of the lateral flaps 38 and 40 may be varied in order to vary the product dimensions and characteristics. For example, in one embodiment, the lateral flaps may have a width that is from about 25% to about 50% of the width of the middle portion 14. In this embodiment, when the lateral flaps are folded, the lateral flaps do not contact each other but, instead, create a channel in the middle portion of the absorbent structure. The channel may be used, for instance, to improve fluid handling characteristics. For instance, the channel may be used to collect fluids prior to the fluids being absorbed by the absorbent material.

In other embodiments, the lateral flaps 38 and 40 may have a width that is greater than 50% of the width of the middle portion, such as having a width from about 50% to 100% of the width of the middle portion 14. In this embodiment, once the lateral flaps 38 and 40 are folded, the flaps overlap to form a three layer structure. Creating a three layer structure further increases the basis weight of the middle portion 14.

In other embodiments, the blank 20 may be made with a non-rectangular shape that would create other basis weight differentials.

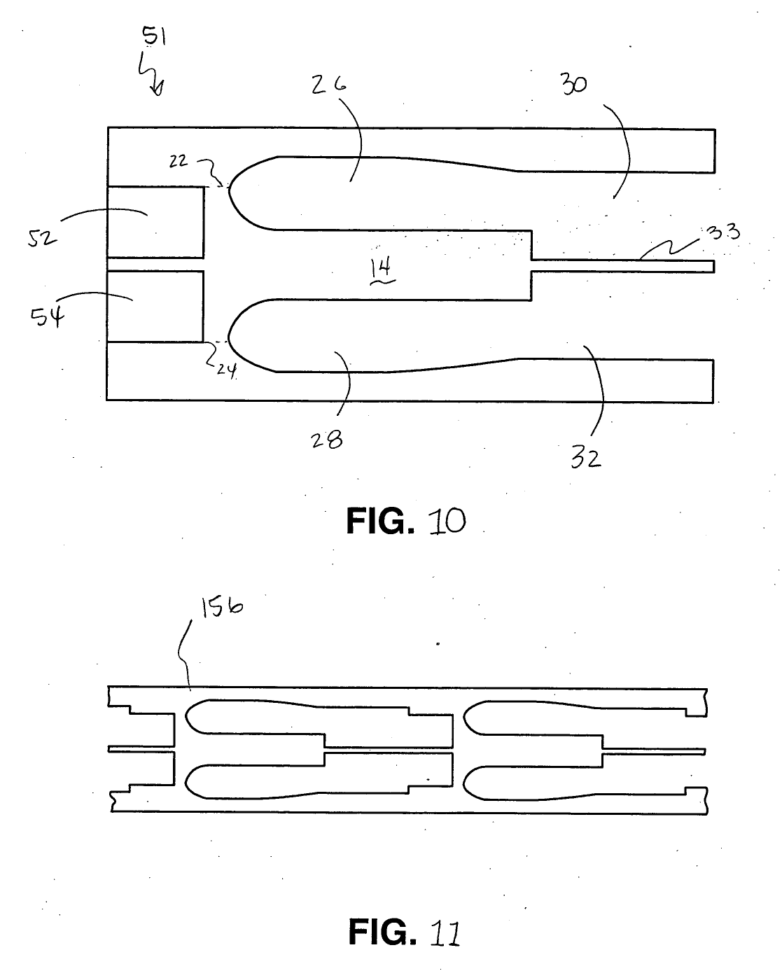

Although the absorbent structure 10 as shown in FIG. 5 may be used in any suitable absorbent product, the absorbent structure 10 is particularly well suited for use in male specific products. In particular, the absorbent structure 10 includes greater liquid absorbent areas in the middle portion and in the front portion where typically needed for a male wearer. Referring to FIGS. 9 and 10, on the other hand, a female specific absorbent structure generally 50 is shown. In FIGS. 9 and 10, like reference numerals have been included in order to identify similar features or areas of the absorbent structure 50.

Referring to FIG. 10, a blank generally 51 is illustrated that may be used to construct the absorbent structure 50 as shown in FIG. 9. In comparison to the blank 20 as shown in FIG. 7, in this embodiment, the blank 51 includes a longer middle portion 14 and includes a pair of opposing front openings 52 and 54.

Referring to FIG. 9, when the lateral flaps 38 and 40 are folded in this embodiment, the higher basis weight area is generally shifted towards the rear portion 16 due to the elongation of the middle portion 14 and the presence of the front openings 52 and 54. For example, as shown, the absorbent structure 50 includes a front portion 12, a middle portion 14, and a rear portion 16. When the lateral flaps 38 and 40 are folded, the front openings 52 and 54 are shown located at the uppermost location of the front portion 12. Thus, the absorbent structure 50 includes a primarily single layer area in the front portion and in the rear portion and a two-layer area in the middle portion 14 and extending partly into the front portion 12. By shifting the higher basis weight areas as shown in FIG. 9, the absorbent structure 50 is better suited for use in absorbent products that are female specific. The overall shape of the absorbent structure 50, however, is substantially the same as the overall shape of the absorbent structure 10 as shown in FIG. 5.

As described above, in other embodiments, the lateral flaps 38 and 40 as shown in FIG. 9 may have a width that is from about 25% to 100% of the width of the middle portion 14. When having a width that is less than 50% of the width of the middle portion, a fluid handling channel forms in the absorbent structure 10. When the lateral flaps have a width that is greater than 50% of the width of the middle portion 14, on the other hand, the lateral flaps overlap and a three layer structure is formed.

As will be described in more detail below, the absorbent structures of the present invention can be constructed from a single layer of material that generally has a uniform basis weight. Through the use of the openings and by folding the lateral flaps, however, basis weight differentials within the product can be formed without creating a substantial amount of trim waste. In fact, in one embodiment, zero trim waste may be produced when forming the absorbent structures. Of particular advantage, gender specific absorbent structures can also be produced by making small changes in the manufacturing process.

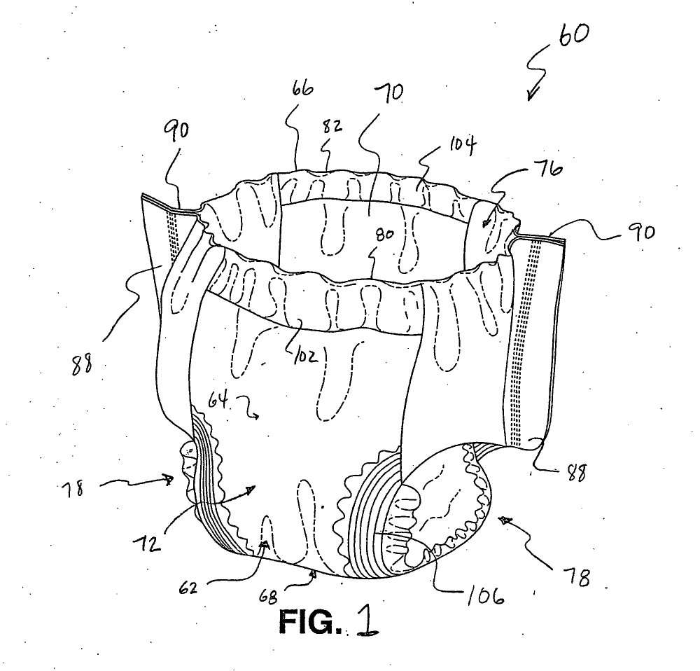

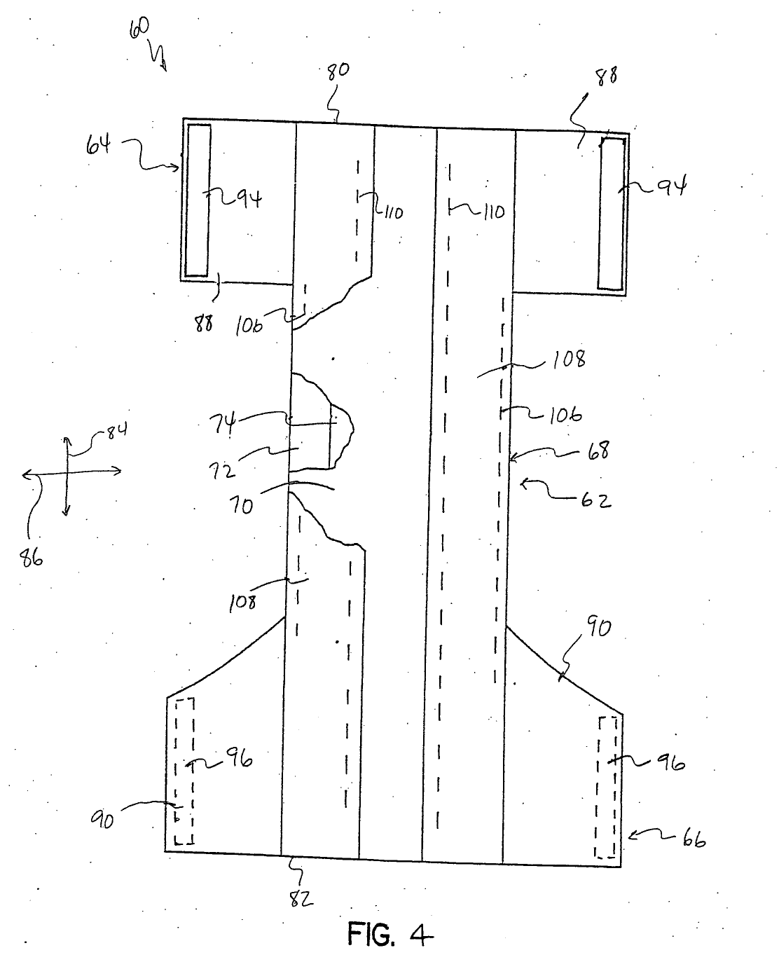

The absorbent structures 10 and 50 as shown in FIGS. 5 and 9 may be incorporated into any suitable absorbent article, such as a diaper, a training pant, an adult incontinence product, a feminine hygiene product, and the like. For example, referring to FIGS. 1-4, a pant-like absorbent article generally 60 is illustrated. The article 60 includes a chassis 62 defining a front region 64, a back region 66, and a crotch region 68 interconnecting the front and back regions. The chassis 62 includes a bodyside liner 70 which is configured to contact the wearer, and an outer cover 72 opposite the bodyside liner which is configured to contact the wearer's clothing. An absorbent structure 74 (see FIG. 4) is positioned or located between the outer cover 72 and the bodyside liner 70. The absorbent structure 74 is made in accordance with the present invention and may be, for instance, an absorbent structure as illustrated in FIG. 5 or an absorbent structure as illustrated in FIG. 9.

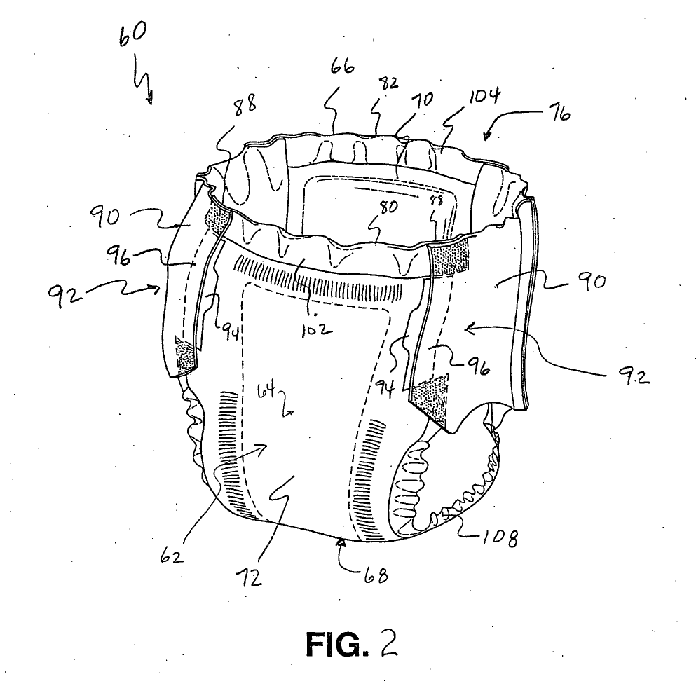

FIG. 2 illustrates an alternative embodiment of an absorbent article 60 similar to the absorbent article illustrated in FIG. 1. Like reference numerals have been used to indicate similar elements. As shown, the absorbent article 60 shown in FIG. 2, different than the embodiment shown in FIG. 1, includes refastenable sides. The absorbent article 60 shown in FIG. 1, on the other hand, has permanently bonded sides. Both embodiments of an absorbent article define a 3-dimensional pant configuration having a waist opening 76 and a pair of leg openings 78. The front region 64 includes the portion of the article 60 which, when worn, is positioned on the front of the wearer while the back region 66 includes the portion of the article which, when worn, is positioned on the back of the wearer. The crotch region 68 of the absorbent article 60 includes the portion of the article which, when worn, is positioned between the legs of the wearer and covers the lower torso of the wearer.

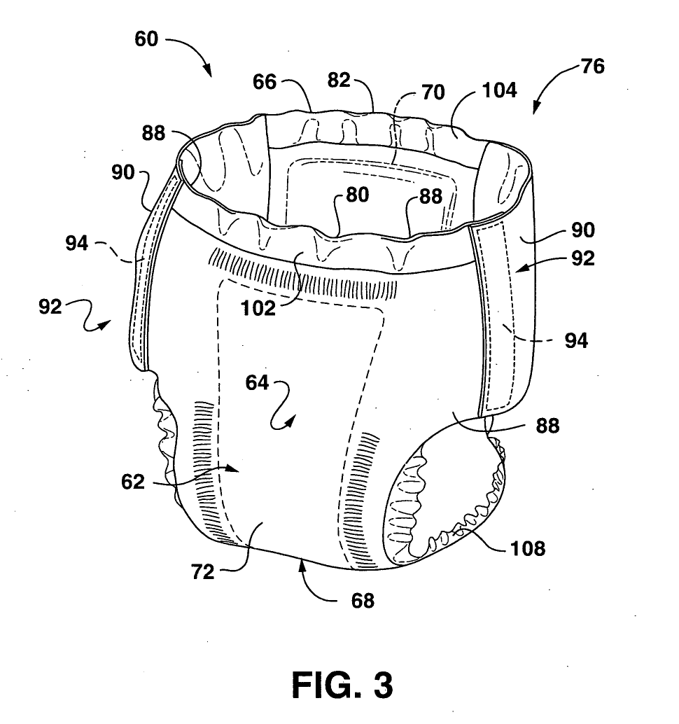

As shown in further detail in FIGS. 1-4, the chassis 62 also defines a pair of longitudinally opposed waist edges which are designated front waist edge 80 and back waist edge 82. The front region 64 is contiguous with the front waist edge 80, and the back region 66 is contiguous with the back waist edge 82. The waist edges 80, 82 are configured to encircle the waist of the wearer when worn and define the waist opening 76. For reference, arrows 84 and 86 depicting the orientation of the longitudinal axis and the transverse axis, respectively, of the absorbent article 60 are illustrated in FIGS. 3 and 4.

The illustrated absorbent chassis 62 includes a pair of transversely opposed front side panels 88, and a pair of transversely opposed back side panels 90. The side panels 88, 90 may be integrally formed with the outer cover 72 and/or the bodyside liner 70 or may include two or more separate elements.

The side panels 88 and 90 suitably include an elastic material capable of stretching in a direction generally parallel to the transverse axis 86 of the absorbent article 60. Suitable elastic materials, as well as processes of incorporating side panels into a training pant, are known to those skilled in the art, and are described, for example, in U.S. Pat. No. 4,940,464 issued Jul. 10, 1990 to Van Gompel et al., which is incorporated herein by reference.

As mentioned, the absorbent article 60 according to the present invention may be refastenable, thereby including a fastening system 92 for securing the training pant above the waist of the wearer (see FIG. 2). The illustrated fastening system 92 may include fastening components 94 that are adapted to refastenably connect to mating fastening components 96. In one embodiment, one surface of each of the fastening components 94 and 96 includes a plurality of engaging elements that project from that surface. The engaging elements of these fastening components 94 are adapted to repeatedly engage and disengage the engaging elements of the mating fastening components 96.

In one particular embodiment, the fastening components 94 each include hook type fasteners and the mating fastening components 96 each include complementary loop type fasteners. In another particular embodiment, the fastening components 94 each include loop type fasteners and the mating fastening components 96 each include complementary hook type fasteners.

As noted previously, the illustrated absorbent article 60 has front and back side panels 88 and 90 disposed on each side of the absorbent chassis 62. These transversely opposed front side panels 88 and transversely opposed back side panels 90 can be permanently bonded to the composite structure comprising the absorbent chassis 62 in the respective front and back regions 64 and 66. Additionally, the side panels 88 and 90 can be permanently bonded to one another using suitable bonding means, such as adhesive bonds or ultrasonic bonds, to provide a non-fastenable absorbent article 60 (FIG. 1). Alternatively, the side panels 88 and 90 can be releaseably attached to one another by a fastening system 92 as described above. More particularly, as shown best in FIG. 3, the front side panels 88 can be permanently bonded to and extend transversely beyond the linear side edges 98 of the composite structure in the front region 64 along attachment lines 100, and the back side panels 90 can be permanently bonded to and extend transversely beyond the linear side edges 98 of the composite structure in the back region 66 along attachment lines 100. The side panels 88 and 90 may be attached using attachment means known to those skilled in the art such as adhesive, thermal or ultrasonic bonding. The side panels 88 and 90 can also be formed as a portion of a component of the composite structure, such as the outer cover 72 and/or the bodyside liner 70.

Each of the side panels 88 and 90 can include one or more individual, distinct pieces of material. In particular embodiments, for example, each side panel 88 and 90 can include first and second side panel portions that are joined at a seam, with at least one of the portions including an elastomeric material. Still alternatively, each individual side panel 88 and 90 can include a single piece of material which is folded over upon itself along an intermediate fold line (not shown). Suitably, the side panels 88 and 90 include an elastic material capable of stretching in a direction generally parallel to the transverse axis 86 of the absorbent article 60.

To enhance containment and/or absorption of body exudates, the absorbent article 60 may include a front waist elastic member 102, a rear waist elastic member 104, and leg elastic members 106, as are all known to those skilled in the art (see FIG. 4). The waist elastic members 102 and 104 can be operatively joined to the outer cover 72 and/or the bodyside liner 70 along the opposite waist edges 80 and 82, and can extend over part or all of the waist edges. The leg elastic members 106 are suitably operatively joined to the outer cover 72 and/or bodyside liner 70 along opposite side edges of the chassis 62 and positioned in the crotch region 68 of the absorbent article 60.

The waist elastic members 102, 104 and the leg elastic members 106 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and attached to a substrate, attached to a gathered substrate, or attached to a substrate and then elasticized or shrunk, for example with the application of heat; such that elastic constrictive forces are imparted to the substrate. In one particular embodiment, for example, the leg elastic members 106 include a plurality of dry-spun coalesced multifilament spandex elastomeric threads sold under the trade name LYCRA and available from E.I. DuPont de Nemours and Co., Wilmington, Del.

To enhance containment and/or absorption of any body exudates discharged from the wearer, the chassis 62 may include a pair of containment flaps 108 which are configured to provide a barrier to the transverse flow of body exudates. A flap elastic member 110 (see FIG. 4) may be operatively joined with each containment flap 108 in any suitable manner as is well known in the art. The elasticized containment flaps 108 define an unattached edge which assumes an upright, generally perpendicular configuration in at least the crotch region 68 of the absorbent article 60 to form a seal against the wearer's body. The containment flaps 108 can be located along the transversely opposed side edges of the chassis 62, and can extend longitudinally along the entire length of the chassis or may only extend partially along the length of the chassis. Suitable constructions and arrangements for the containment flaps 108 are generally well known to those skilled in the art.

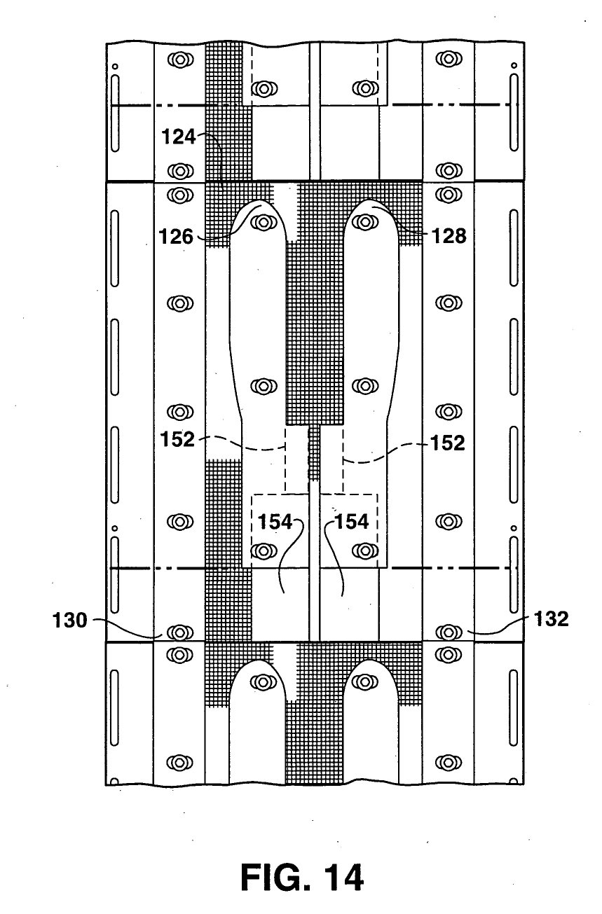

The absorbent articles 60 as shown in FIGS. 14 can be made from various materials. The outer cover 72 may be made from a material that is substantially liquid and permeable, and can be elastic, stretchable or nonstretchable. The outer cover 72 can be a single layer of liquid and permeable material, or may include a multi-layered laminate structure in which at least one of the layers is liquid and permeable. For instance, the outer cover 72 can include a liquid permeable outer layer and a liquid and permeable inner layer that are suitably joined together by a laminate adhesive.

For example, in one embodiment, the liquid permeable outer layer may be a spunbond polypropylene nonwoven web. The spunbond web may have, for instance, a basis weight of from about 15 gsm to about 25 gsm.

The inner layer, on the other hand, can be both liquid and vapor impermeable, or can be liquid impermeable and vapor permeable. The inner layer is suitably manufactured from a thin plastic film, although other flexible liquid impermeable materials may also be used. The inner layer prevents waste material from wetting articles such as bedsheets and clothing, as well as the wearer and caregiver. A suitable liquid impermeable film may be a polyethylene film having a thickness of about 0.2 mm.

A suitable breathable material that may be used as the inner layer is a microporous polymer film or a nonwoven fabric that has been coated or otherwise treated to impart a desired level of liquid impermeability. Other “non-breathable” elastic films that may be used as the inner layer include films made from block copolymers, such as styrene-ethylene-butylene-styrene or styrene-isoprene-styrene block copolymers.

As described above, the absorbent structure is positioned in between the outer cover and a liquid permeable bodyside liner 70. The bodyside liner 70 is suitably compliant, soft feeling, and non-irritating to the wearer's skin. The bodyside liner 70 can be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner 70. For example, the bodyside liner can be made from a meltblown or spunbonded web of polyolefin fibers. The bodyside liner can also be a bonded-carded web composed of natural and/or synthetic fibers.

A suitable liquid permeable bodyside liner 70 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular embodiment, the polypropylene forms the core and the polyethylene forms the sheath of the fiber. Other fiber orientations, however, are possible.

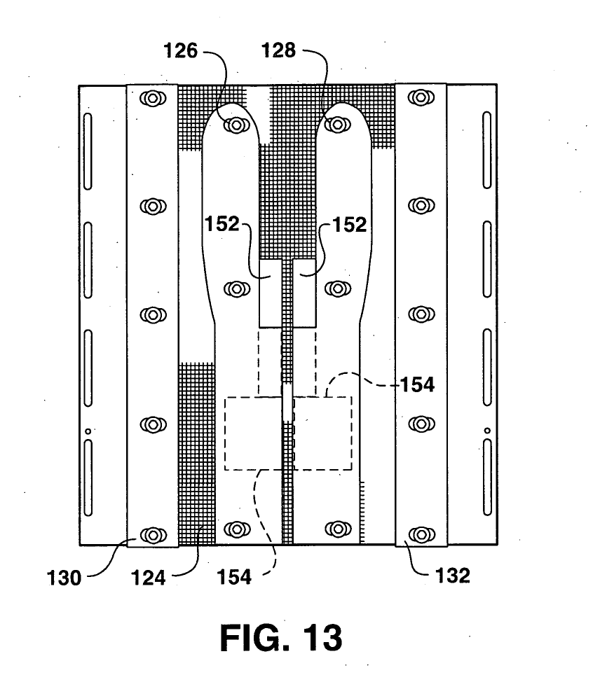

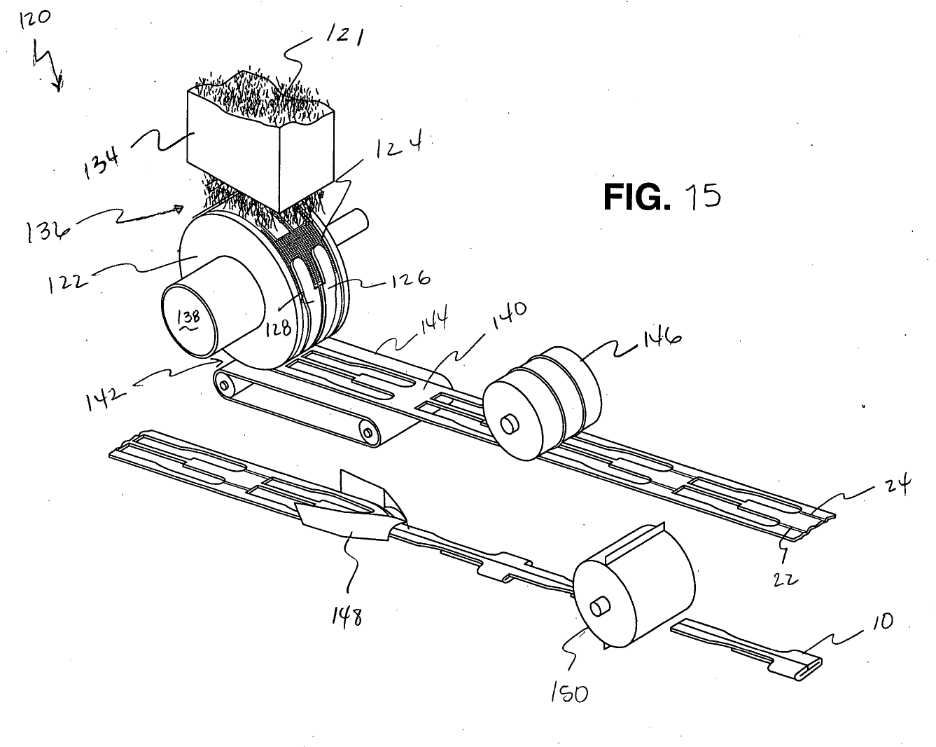

One embodiment of a process for forming absorbent structures in accordance with the present invention will now be described with particular reference to FIGS. 13, 14 and 15. As described above, the absorbent structures may be formed according to various different processes. Referring to FIG. 15, one embodiment of an air forming process generally 120 in accordance with the present invention is shown. The air forming process 120 as shown in FIG. 15 is generally referred to also as a drum forming process. As shown, the system includes a forming drum 122 as particularly illustrated in FIG. 12. The forming drum 122 includes a porous forming surface 124. As shown, the forming surface 124 may comprise a screen. Secured to the forming surface 124 are a pair of masking members 126 and 128 in accordance with- the present invention. The masking members 126 and 128 cause the middle openings and the rear openings to be formed in an absorbent web.

The masking members 126 and 128 are more particularly shown in FIG. 13 secured to the forming surface 124. As shown particularly in FIG. 13, vertical masking strips 130 and 132 may also be secured to the forming surface. The vertical masking strips 130 and 132 are for adjusting the width of the absorbent web that is formed.

As shown in FIG. 15, in order to form an absorbent web, a selected fibrous material 121 can be introduced into the system as air-entrained fibers in a stream flowing in the direction toward the forming surface 124. The fibers may suitably be derived from a batt of cellulosic fibers (e.g., wood pulp fibers) or other source of natural or synthetic fibers, which has been subjected to a fiberization treatment, in a manner well known in the art, to provide an operative quantity of individual, loose fibers. For example, a hammer mill or other conventional fiberizer may be employed. Particles or fibers of superabsorbent material may also be introduced into a forming chamber 134 by employing conventional mechanisms, such as pipes, channels, spreaders, nozzles and the like, as well as combinations thereof. The fibers and particles may be entrained in any suitable gaseous medium, and references herein to air as being the entraining medium should be understood to be a general reference which encompasses any other operative entrainment gas.

The stream of air-entrained fibers and particles can pass through the forming chamber 134 and onto the forming surface 124 of the forming drum 122. The forming chamber can serve to direct and concentrate the air-entrained fibers and particles, and to provide a desired velocity profile in the air-entrained stream of fibers and particles. The forming chamber is typically supported by suitable structural members, which together form a support frame for the forming chamber.

As shown, the forming drum 122 is rotatable in a selected direction of rotation, and can be rotated by employing a drum drive shaft that is operatively joined to any suitable drive mechanism (not shown). For example, the drive mechanism can include an electric or other motor which is directly or indirectly coupled to the drive shaft. While the shown arrangement provides a forming drum that is arranged to rotate in a counter-clockwise direction, it should be readily apparent that the forming drum may alternatively be arranged to rotate in a clockwise direction.

The forming drum 122 can provide a laydown zone 136 which is positioned within the forming chamber 134 and provides a vacuum laydown zone of the foraminous forming surface 124. This vacuum laydown zone constitutes a circumferential, cylindrical surface portion of the rotatable drum 122. An operative pressure differential is imposed on the surface of the vacuum laydown zone under the action of a conventional vacuum generating mechanism, such as a vacuum pump, an exhaust blower or other suitable mechanism which can provide a relatively lower pressure under the forming surface 124. The vacuum mechanism can operatively withdraw air from the arcuate segment of the forming drum associated with the vacuum laydown surface through an air discharge duct 138.

As shown, the foraminous forming surface 124 can include a series of forming sections which are distributed circumferentially along the periphery of the forming drum 122. The succession of forming sections can provide a selected repeat pattern that is formed into a fibrous web. For example, as shown, four sets of masking members 126 and 128 are shown around the circumference of the forming drum 122.

Suitable forming drum systems for producing air laid fibrous webs are known. For example, U.S. Pat. Nos. 4,666,647, 4,761,258, 6,330,735 and 4,927,582 all disclose air forming processes and all are incorporated herein by reference.

Thus, under the influence of a vacuum mechanism, a conveying air stream is drawn through the foraminous forming surface 124 into the interior of the forming drum 122, and is subsequently passed out of the drum through the discharge duct 138. As the air entrained fibers and particles impinge on the forming surface 124, the air component thereof is passed through the forming surface and the fibers-particles component is retained on the forming surface to form a commingled fibrous web or pad 140 thereon. In accordance with the present invention, the masking members 126 and 128 prevent the formation of a web on the forming surface 124 where they are located. Thus, through the use of the masking members 126 and 128, openings are formed in the web at particular locations for forming absorbent structures in accordance with the present invention.

Subsequently, with the rotation of the drum 122, the formed web 140 can be removed from the forming surface 124 by the weight of the web, by centrifugal force, and by a positive pressure produced, for example, by a pressurized air flow through a blow off zone 142. The pressurized air can exert a force directed outwardly through the forming surface.

As shown in FIG. 15, a continuous strip of the fibrous web 140 is produced and is conveyed by a conveyor 144 in a machine direction. A top plan view of the strip of absorbent web material 140 that is formed is shown in FIG. 8. As illustrated, a series or succession of blanks 20 as shown in FIG. 8 are produced by the drum forming apparatus. Each blank includes a front portion 12, a middle portion 14, and a rear portion 16. Further, the masking members 126 and 128 form the middle openings 26 and 28 and the rear openings 30 and 32. As particularly shown in FIG. 8, the thin strip of material 33 separating the rear openings 30 and 32 provides integrity to the continuous strip of material as the material is moved and processed.

Referring back to FIG. 15, the produced strip of web material 140 is then fed to a scoring and debulking apparatus 146. The scoring and debulking apparatus 146 comprises a roll that includes raised portions that form the scoring lines 22 and 24. The apparatus 146 also debulks at least portions of the web. For example, in one embodiment, the apparatus compresses and densifies the lateral flaps.

From the scoring and debulking apparatus 146, the absorbent web of material 140 is then fed to a folding device 148 which folds the lateral flaps along the scoring lines 22 and 24. Next, the web of material 140 is fed to a cutting device 150. The cutting device 150 cuts the material 140 in the cross-machine direction in order to form individual absorbent structures for incorporation into various absorbent products.

As described above, the masking members 126 and 128 as shown in FIG. 13 may be used to produce an absorbent structure 10 as shown in FIG. 5. The absorbent structure 10 is particularly well suited for use in male specific products. One of the advantages of the process and system of the present invention is the ability to easily switch from the production of a male specific product to a female specific product and vice versa. In this regard, as shown in FIG. 13, each of the masking members 126 and 128 can include a movable middle tab 152 and a movable rear tab 154 (shown in phantom). The middle tabs 152 and the rear tabs 154 may be movable on the masking members as shown in FIG. 13 or, alternatively, may be easily removed altogether from the masking members. Further, although the middle tabs 152 and the rear tabs 154 are shown in a 2-piece construction, it should be understood that the masking members can include a single middle tab 152 and a single rear tab 154.

As described above, when changing from a male specific absorbent structure to a female specific absorbent structure, it is generally desirable to move the higher basis weight area more towards the middle and rear of the absorbent structure. According to the present invention, the middle tabs 152 and the rear tabs 154 allow for shifting of the higher basis weight area when forming absorbent structures. For example, referring to FIG. 14, the forming surface 124 is shown in which the masking members 126 and 128 have been converted in order to form female specific absorbent structures. As shown, the middle tabs 152 as illustrated in FIG. 13 have been retracted within each masking member 126 and 128. Alternatively, as described above, the middle tabs 152 may also be completely removed from each masking member.

As also shown, the rear tabs 154 have been placed in an extended position. As shown in FIG. 10 and 14, the rear tabs 154 actually produce the front openings 52 and 54 in an absorbent structure made with the forming surface 124.

Any suitable device or mechanism may be used in order to retract and extend the middle tabs 152 and the rear tabs 154. For example, the tabs may slide below or over top of each masking member 126 and 128 when it is desired either to hide the tabs or extend the tabs. In this embodiment, the tabs may be removably tightened against each masking member using, for instance, a suitable bolt or screw. By loosening the bolt or screw, the tabs 152 and 154 may be easily slid into an extended position or a retracted position.

Alternatively, the tabs 152 and 154 may be attached to the masking members 126 and 128 with hinges. In this manner, the tabs 152 and 154 may swing or pivot between a retracted position and an extended position. It should be understood, however, that various other means and mechanisms may be used in order to retract or extend the tabs. Also, as stated above, the tabs may be completely removable from the masking members.

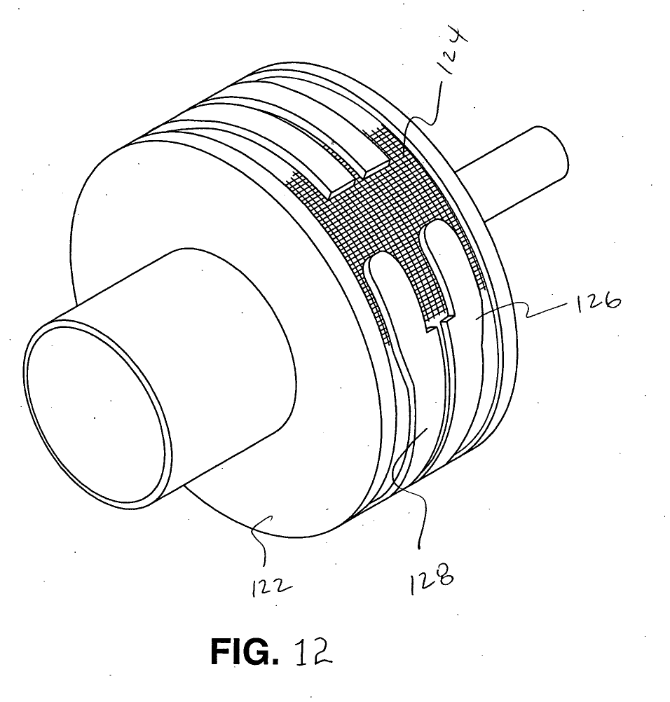

As shown in FIG. 14, by retracting the middle tabs 152 and extending the rear tabs 154 a blank 51 may be produced as shown in FIG. 10 for forming the absorbent structure 50 as shown in FIG. 9. By retracting the tabs 152 and extending the tabs 154, the higher basis weight area is shifted from the front towards the middle portion of the absorbent pad. When located on a forming drum in a repeating pattern, the masking members as shown in FIG. 14 may be used to form a continuous strip of absorbent material 156 as shown in FIG. 11.

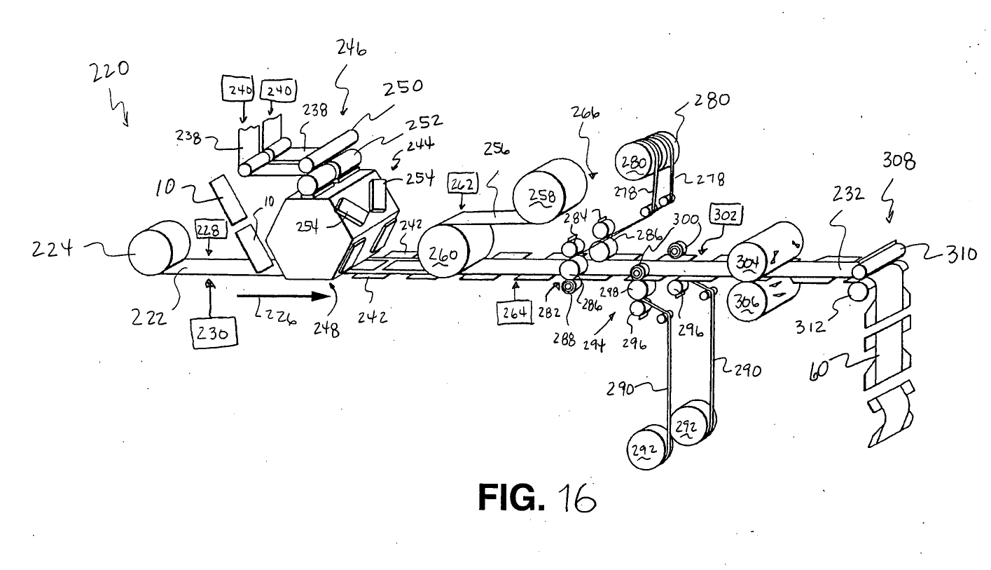

Referring now to FIG. 16, an exemplary embodiment of an assembly section 220 for making a continuous stream of partially assembled, discrete pants or garments 60 is illustrated. The specific equipment and processes used in the assembly section 220 can vary greatly depending on the specific type of garment being manufactured. The particular process and apparatus described in relation to FIG. 16 is specifically adapted to manufacture absorbent articles 60 of the type illustrated in FIGS. 1 through 4.

The various components of the garment 60 can be connected together by any means known to those skilled in the art such as, for example, adhesive, thermal and/or ultrasonic bonds. Suitably, most of the components are connected using ultrasonic bonding for improved manufacturing efficiency and reduced raw material costs. Certain garment manufacturing equipment which is readily known and understood in the art, including frames and mounting structures, ultrasonic and adhesive bonding devices, transport conveyors, transfer rolls, guide rolls, tension rolls, and the like, have not been shown in FIG. 16.

A continuous supply of material 222 used to form the bodyside liner 70 is provided from a supply source 224. The supply source 224 can include for example a pair of spindles, a festoon assembly, and optionally a dancer roll (not shown) for providing bodyside liner material 222 at a desired speed and tension.

Various components can be disposed on and/or bonded to the bodyside liner material 222 as the material travels in a machine direction identified by arrow 226. In particular, a surge layer can be provided at an application station 228 and disposed on and/or bonded to the bodyside liner material 222. The surge layer can include either a continuous web or discrete sheets.

Surge layers are generally well known in the art as being constructed to quickly collect and temporarily hold liquid surges, and to transport the temporarily held liquid to the absorbent structure 10.

Various woven and non-woven fabrics can be used to construct the surge layer. For example, the surge layer may be a layer made of a meltblown or spunbond web of synthetic fibers, such as polyolefin fibers. The surge layer may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge layer may be composed of a substantially hydrophobic material, and the hydrophobic material may optionally be treated with a surfactant or otherwise processed to impart a desired level of wettability and hydrophilicity.

Examples of materials suitable for the surge layer are set forth in U.S. Pat. No. 5,486,166 issued Jan. 23, 1996 in the name of C. Ellis et al. and entitled “FIBROUS NONWOVEN WEB SURGE LAYER FOR PERSONAL CARE ABSORBENT ARTICLES AND THE LIKE”; U.S. Pat. No. 5,490,846 issued Feb. 13, 1996 in the name of Ellis et al. and entitled “IMPROVED SURGE MANAGEMENT FIBROUS NONWOVEN WEB FOR PERSONAL CARE ABSORBENT ARTICLES AND THE LIKE”; and U.S. Pat. No. 5,364,382 issued Nov. 15, 1994 in the name of Latimer et al. and entitled “ABSORBENT STRUCTURE HAVING IMPROVED FLUID SURGE MANAGEMENT AND PRODUCT INCORPORATING SAME”, the disclosures of which are hereby incorporated by reference in a manner consistent with the present document.

Additionally, a containment flap module 230 can be provided downstream of the supply source 224 for attaching pre-assembled containment flaps to the bodyside liner material 222. The containment flaps are located generally adjacent to the side edges of the garment assembly 60, and can extend longitudinally along the entire length of the garment assembly 60 or only partially along the length of the garment assembly. Suitable constructions and arrangements for the containment flaps are generally well known to those skilled in the art and are described in U.S. Pat. No. 4,704,116 issued Nov. 3, 1987 to Enloe, which is incorporated herein by reference.

As various components are added in the assembly section 220, a continuously moving product assemblage 232 is formed. The product assemblage 232 will be cut downstream to form the partially assembled, discrete garments 60.

A plurality of absorbent structures 10 are provided from a suitable supply source. The supply source can be, for instance, the air forming system and process as shown in FIG. 15.

Assembly section 220 can include a device to apply side panels. For example, continuous webs of material 238 used to form the side panels 88 and 90 can be provided from suitable supply sources 240. The supply sources 240 can include one or more unwind mechanisms. The side panel material 238 can be cut into individual strips 242 and positioned partially on the bodyside liner material 222 using an applicator device 244. In the cross machine direction, the individual strips 242 suitably extend laterally outward from the bodyside liner material 222 and overlap the bodyside liner material to permit bonding of the strips to the bodyside liner and/or the containment flap material. Bonding may be accomplished using adhesives, as is well known in the art, or by any other bonding means. In the machine direction 226, the position of the strips 242 can be registered relative to the absorbent assemblies 10 so that the product assemblage 232 can be cut between the absorbent assemblies with each strip 242 of side panel material 238 forming both a front side panel 88 and a back side panel 90 of consecutive garments 60.

One suitable applicator device 244 is disclosed in U.S. Pat. No. 5,104,116 issued Apr. 14, 1992 and U.S. Pat. No. 5,224,405 issued Jul. 6, 1993 both to Pohjola, which are incorporated herein by reference. The applicator device 244 can include a cutting assembly 246 and a rotatable transfer roll 248. The cutting assembly 246 employs a rotatable knife roll 250 and a rotatable vacuum anvil roll 252 to cut individual strips 242 from the continuous side panel material 238. The strips 242 cut by a blade on the knife roll 250 can be maintained on the anvil roll 252 by vacuum and transferred to the transfer roll 248.

The rotatable transfer roll 248 can include a plurality of rotatable vacuum pucks 254. The vacuum pucks 254 receive the strips 242 of material 238 from the cutting assembly 246 and rotate and transfer the strips to the continuously moving bodyside liner material 222. When the strips 242 are positioned as desired relative to the bodyside liner material 222, the strips are released from the pucks 254 by extinguishing the vacuum in the pucks. The pucks 254 can continue to rotate toward the cutting assembly 246 to receive other strips.

Alternative configurations for attaching the side panel material 238 exist. For instance, the material 238 used to form the side panels can be provided in continuous form and contour cut to form leg openings 78. Still alternatively, the side panels 88 and 90 of the pant 60 can be provided by portions of the bodyside liner 70 and/or outer cover 72. It should be noted that the side panel application processes just described are exemplary only, and that the process can vary greatly depending on the physical characteristics of the material and the nature of the process.

A continuous supply of material 256 used to form the outer cover 72 can be provided from a supply roll 258 or other suitable source. As the material is unwound, the outer cover material 256 can be married with the bodyside liner material 222 such as by use of a laminator roll 260. The absorbent assemblies 10 are thereby sandwiched between the continuous materials 222 and 256. The inward portions of the strips 242 of side panel material 238 can also be disposed between the bodyside liner material 222 and the outer cover material 256. Various components such as leg elastics 106 or waist elastics 102 and 104 can be bonded to the outer cover material 256 at an application station 262 prior to uniting the bodyside liner and outer cover materials 222 and 256. Alternatively, leg elastics or waist elastics can be initially bonded to the bodyside liner material 222 or another material.

The outer cover 256 can be joined to the liner-side panel composite using any means known to those of skill in the art. Where an adhesive is used, the adhesive can be applied on or prior to laminator roll 260. Alternatively, bonding devices such as ultrasonic or thermal bonders can be employed as part of the laminator roll 260 or at a downstream location 264 to bond the bodyside liner material 222, side panel material 238 and outer cover material 256.

The assembly section 220 can include apparatus to provide/apply a fastening system to the garment 60. For example, the continuously moving product assemblage next advances to a fastener application station 266 where fastening components 94 and 96 can be bonded to the strips 242 of side panel material 238. The location of the fastening components on the composite is a function in part of the configuration of the assembly section 220. The illustrated assembly section 220 is configured so that the upwardly facing surface of the product assemblage 232 will become the outer surface of the pant 60 and the downwardly facing surface will become the inner surface. Moreover, the illustrated assembly section 220 is configured to produce partially assembled garments 60 having the front waist region 64 of a leading garment connected to the back waist region 66 of a trailing garment. The process could alternatively employ any combination of different orientations. For example, the upwardly facing surface of the product assemblage could form the inner surface of finished garments. Additionally or alternatively, the back waist region 66 of a leading garment can be connected to the front waist region 64 of the trailing garment, or the garments can be arranged in a front-to-front/back-to-back relationship. Still alternatively, the assembly section 220 can be constructed as a cross-machine direction process wherein the longitudinal axis of each garment could be perpendicular to the machine direction 226 during part or all of the assembly process.

Continuous webs of a fastener material 278 used to form the fastening components 96 (FIGS. 2 and 4) can be provided from supply rolls 280 or other suitable sources. The fastener materials 278 can be cut into individual fasteners 96 by cutting assemblies 282 or other suitable devices. The illustrated cutting assemblies 282 include rotatable knife rolls 284, rotatable vacuum anvil rolls 286, and rotatable backing rolls 288. The continuous fastener materials 278 can be cut by blades on the knife rolls 284, maintained on the anvil rolls 286 by vacuum, and disposed on the top surfaces of the strips 242 of side panel material 238.

Similarly, continuous webs of a fastener material 290 used to form the fastening components 94, shown in FIGS. 2 and 4, can be provided from supply rolls 292 or other suitable sources. The first fastener materials 290 can be cut into individual first fasteners 94 by cutting assemblies 294 or other suitable devices.

Alternatively, a component of the garment 60 may serve as the fastening components, in which case some or all of the fastener application station 266 or the cutting assemblies 294 may not be needed. The illustrated cutting assemblies 294 include rotatable knife rolls 296, rotatable vacuum anvil rolls 298, and rotatable backing rolls 300. The continuous fastener materials 290 can be cut by blades on the knife rolls 296, maintained on the anvil rolls 298 by vacuum, and disposed on the undersides of the strips 242 of side panel material 238.

Other arrangements can be used to attach the fastening components 94 and 96. For example, the fastening components can be applied to the side panel material 238 prior to uniting the side panel material with the bodyside liner material 222 and/or the outer cover material 256; the fastening components can be applied to the bodyside liner material 222 and/or outer cover material 256, whether separate side panels are used or not; portions of other components such as the bodyside liner and/or outer cover can form one or more of the fastening components; the separate side panels or integral side panels can themselves form one or more of the fastening components; the fastening components can be attached as pre-engaged composites; or the like.

After the fastening components are disposed on the strips 242 of side panel material 238, bonding devices 302 such as ultrasonic bonders can be employed to bond the fastening components to the strips. For example, the strips 242 can be transported between a rotary ultrasonic horn and an anvil roll, which devices are positioned on each side of the process at the cross machine direction location of the fastening components 94 and 96. Particular ultrasonic bond patterns including individual, circular bonds which are compatible with mechanical fastening materials are disclosed in U.S. Pat. No. 5,660,666 issued Aug. 26, 1997 to Dilnik et al., which is incorporated herein by reference. Efficient arrangements for attaching the fastening components with nonadhesive bonding devices are further described in U.S. Pat. No. 6,562,167, issued May 13, 2003 by J. D. Coenen et al. and titled “Methods For Making Garments With Fastening Components”, which is incorporated herein by reference. For secure attachment, it may be desirable to attach the fastening components with both adhesive and thermal bonds. Suitable attachment adhesives are available from commercial vendors such as Findley Adhesive, Inc., Wauwatosa, Wis. U.S.A.

In particular embodiments, the bonding devices 302 can provide timed, non-uniform bonding of the fastening components to the side panel material 238. The degree of bonding, such as the number of bonds per unit area or the bond strength per unit area, can be greater in certain target areas compared to non-target areas. Enhanced bonding in target areas can be beneficial particularly near the waist and leg openings to reduce or prevent delamination of the fastening components from the side panel material 238. Thus, the bonding devices 302 can be adapted to create relatively more bonds or stronger bonds between the fastening components and the side panel material 238 when the side panel material 238 reaches a particular machine direction 226 location. In one particular embodiment, the target areas correspond to portions of the fastening components 94 and 96 near the waist edges 80 and 82. The bonding devices 302 can be registered to provide a relatively higher degree of bonding which begins while disposed on one fastening component, continues through the region where the product assemblage 232 will subsequently be cut, and ends after being disposed on another fastening component. Alternatively, the bonding devices 302 can destroy engaging elements of the fastening components in the target areas, so that the fastening components will be less able to aggressively attach to one another in the target areas.

The strips 242 of side panel material 238 can be trimmed if desired, for example to provide angled and/or curved leg end edges in the back and/or front waist regions. To this end, the assembly section 220 can include a die cutting roll 304 and a backing roll 306. In the illustrated embodiment, a portion of each strip 242 is trimmed from a trailing edge in order to form the angled and/or curved leg end edges in the back waist region.

The method and apparatus to this point provides a continuous web of interconnected and partially assembled pants moving in the direction indicated by arrow 226. This continuously moving product assemblage 232 is passed through a cutter 308 which selectively cuts the web into discrete, partially assembled garments 60. Such cutters 308 are generally known to those skilled in the art and can include, for example, the combination of a cutting roll 310 and an anvil roll 312 through which the web travels. The anvil roll 312 can include a hardened steel rotating roll while the cutting roll 310 can include one or more flexible hardened steel blades clamped onto another rotating roll. The pinching force between the blade on the cutting roll 310 and the anvil roll 312 creates the cut. The cutting roll 310 can have one or more blades depending upon the desired distance between the cuts. The cutter 308 can further be configured to provide a spacing between the individual cut pieces after they are cut. Such a spacing can be provided by transferring the cut pieces away from the cutter at a higher speed than the speed at which the web is provided to the cutter.

The discrete garments 60 can then be folded and packaged as desired.

It should be understood that the absorbent structure 10 as shown in FIG. 5 and the absorbent structure 50 as shown in FIG. 9 represent exemplary embodiments of absorbent structures that may be made in accordance with the present invention and incorporated into a garment 60. The teachings and principles of the present invention may be used to form many different absorbent structures in many different configurations.

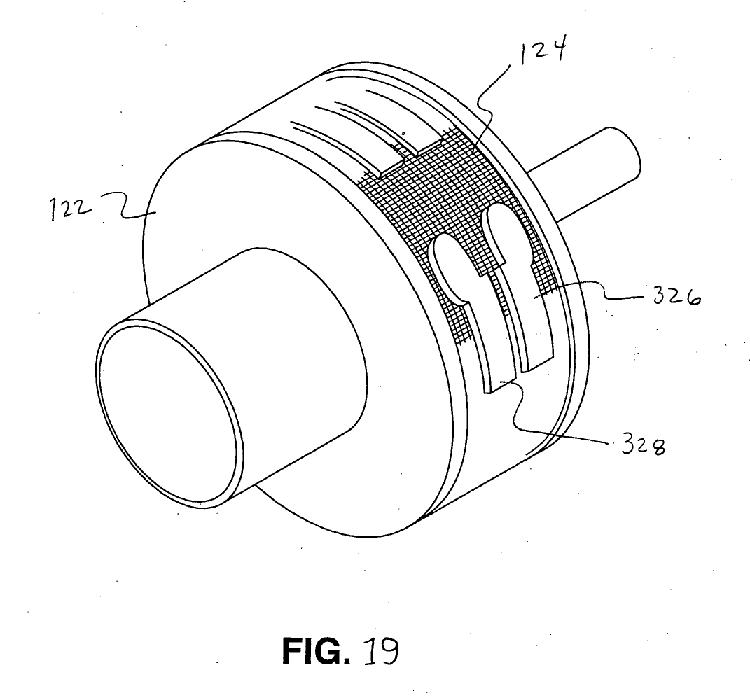

For example, referring to FIG. 18, another embodiment of an absorbent structure 320 made in accordance with the present invention is shown. As illustrated, the absorbent structure 320 includes a front portion 330, a middle portion 332, and a rear portion 334. In this embodiment, the rear portion 334 is narrower than the front portion 330.

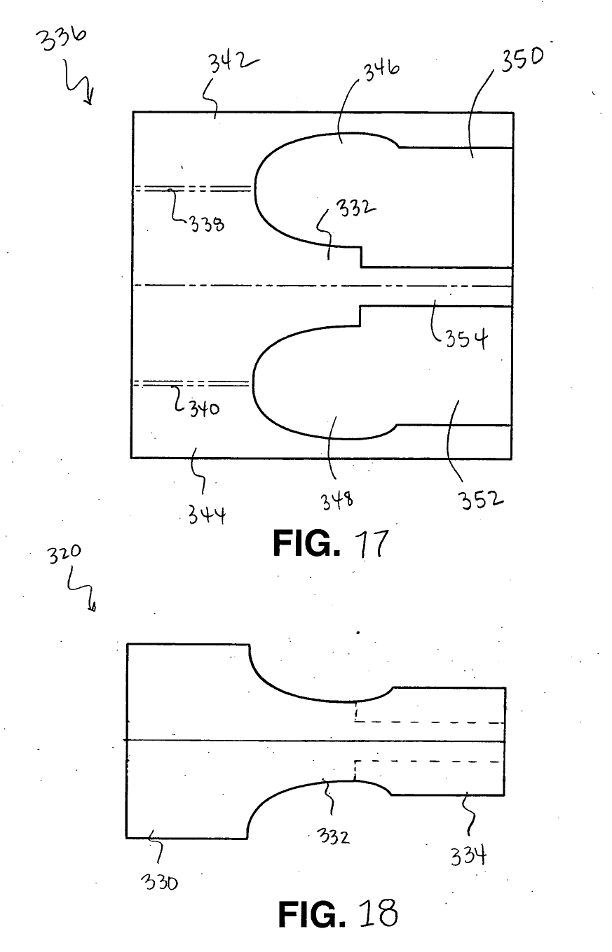

A blank generally 336 is shown in FIG. 17 which may be used to form the absorbent structure 320. As shown, the blank 336 includes a pair of score lines 338 and 340 which define lateral flaps 342 and 344. The blank 336 further includes a pair of middle openings 346 and 348 and a pair of rear openings 350 and 352. The rear openings 350 and 352 are interconnected to the middle openings 346 and 348. Further, the rear openings 350 and 352 are separated by a strip of material 354. The strip of material 354 is for providing integrity to the blank when present in a continuous supply of material containing a succession of repeating blanks 336.

When the lateral flaps 342 and 344 are folded as shown in FIG. 18, the front portion 330 and the middle portion 332 each have a basis weight that is at least about twice the basis weight of most of the rear portion 334.

Referring to FIG. 19, a forming drum 122 is shown having a forming surface 124 that may be used to form the blank 336 as shown in FIG. 17. In particular, attached to the forming surface 124 are a pair of masking members 326 and 328 which form the middle openings 346 and 348 and the rear openings 350 and 352 as shown in FIG. 17.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. An absorbent article comprising:
an outer cover material;
a liner; and
an absorbent structure positioned between the outer cover material and the liner, the absorbent structure comprising:
(a) a front portion;
(b) a middle portion;
(c) a rear portion;
(d) a pair of opposing lateral flaps;
(e) a pair of opposing middle openings spaced between the middle portion and the lateral flaps;
(f) at least one rear opening; and
wherein the lateral flaps have been folded onto at least the middle portion, the middle portion having a width narrower than the width of the front portion due to the location of the pair of opposing middle openings, the folded lateral flaps creating at least a two layer structure in the location of the middle portion.

2. An absorbent article as defined in claim 1, wherein the folded lateral flaps create a basis weight in the location of the middle portion that is at least about twice the basis-weight of areas of the rear portion.

3. An absorbent article as defined in claim 1, wherein the middle openings have an inner concave-shaped edge and an outer convex-shaped edge that cooperate when the lateral flaps are folded to give the absorbent structure an overall hourglass-like shape.

4. An absorbent article as defined in claim 1, wherein the absorbent structure includes two opposing rear openings separated by a strip of material, the strip of material being connected to the middle portion and narrower than the middle portion.

5. An absorbent article as defined in claim 4, wherein each of the rear openings is connected to a corresponding middle opening.

6. An absorbent article as defined in claim 1, wherein the lateral flaps are adhesively secured to the middle portion.

7. An absorbent article as defined in claim 1, wherein the absorbent structure has a length and wherein the lateral flaps extend substantially the entire length of the absorbent structure.

8. An absorbent article as defined in claim 1, wherein the absorbent structure has a rectangular shape when placed in an unfolded state.

9. An absorbent article as defined in claim 1, wherein the absorbent structure includes score lines that separate and define the lateral flaps.

10. An absorbent article as defined in claim 1, wherein the middle portion has a basis weight that is at least about twice the basis weight of areas of the front portion.

11. An absorbent article as defined in claim 2, wherein the folded lateral flaps create a basis weight in the location of the front portion that is at least about twice the basis weight of areas of the rear portion.

12. An absorbent article as defined in claim 1, wherein the folded lateral flaps create a basis weight in the location of the middle portion that is at least 25% greater than the basis weight of areas of the rear portion.

13. An absorbent article as defined in claim 1, wherein the folded lateral flaps create a basis weight in the location of the middle portion that is at least 150% greater than the basis weight of areas of the rear portion.

14. An absorbent article as defined in claim 1, wherein the absorbent structure has a generally uniform basis weight when placed in an unfolded state, except for the location of the middle openings and the at least one rear opening.

15. An absorbent article as defined in claim 1, wherein the absorbent article is one of diapers, child's training pants, feminine care articles, and incontinence articles.

16. An absorbent article as defined in claim 1, wherein the absorbent structure comprises pulp fibers and superabsorbent particles.

17. An absorbent article as defined in claim 1, wherein the absorbent structure comprises an air formed web.

18. An absorbent article as defined in claim 11, wherein the basis weight of the absorbent structure is from about 100 gsm to about 2000 gsm.

19. An absorbent article as defined in claim 1, wherein the absorbent structure has been debulked and has a density of from about 0.1 g/cc to about 0.45 g/cc.

20. An absorbent article as defined in claim 1, wherein the absorbent structure contains synthetic binder fibers.

21. An absorbent article as defined in claim 1, wherein the absorbent structure contains an adhesive.

22. A method of forming absorbent pads from an absorbent web material comprising:
forming a strip of an absorbent web material, the strip of absorbent web material including a succession of interconnected individual absorbent pads, each of the pads including:
(a) a front portion;
(b) a middle portion;
(c) a rear portion;
(d) a pair of opposing lateral flaps;
(e) a pair of opposing middle openings spaced between the middle portion and the lateral flaps; and
(f) at least one rear opening;
conveying the strip of absorbent web material in a machine direction;
folding the opposing lateral flaps onto at least the middle portion of each individual absorbent pad; and
cutting the strip of web material in a cross direction into the individual absorbent pads, wherein the middle portion has a width narrower than the width of the front portion due to the location of the pair of opposing middle openings, once folded, the lateral flaps creating at least a two layer structure in the location of the middle portion.

23. A method as defined in claim 22, wherein by folding the lateral flaps, the lateral flaps create a basis weight in the location of the middle portion of each absorbent pad that is at least about twice the basis weight of areas of the rear portion.

24. A method as defined in claim 22, wherein the middle openings have an inner concave-shaped edge and an outer convex-shaped edge that cooperate when the lateral flaps are folded to give the absorbent structure an overall hourglass-like shape.

25. A method as defined in claim 22, wherein the absorbent structure includes two opposing rear openings separated by a strip of material, the strip of material being connected to the middle portion and being narrower than the middle portion.

26. A method as defined in claim 25, wherein each of the rear openings is connected to a corresponding middle opening.

27. A method as defined in claim 22, wherein the strip of absorbent web material is air formed.

28. A method as defined in claim 22, wherein the strip of the absorbent web material comprises pulp fibers and superabsorbent particles.

29. A method as defined in claim 27, wherein the pair of opposing middle openings and the at least one rear opening are formed during the air forming process.

30. A method as defined in claim 22, wherein the pair of opposing middle openings and the at least one rear opening are formed by cutting the absorbent web material.

31. A method as defined in claim 22, further comprising the step of applying an adhesive to the strip of the absorbent web material in order to adhere the lateral flaps to the web material.

32. A method as defined in claim 22, further comprising the step of scoring the absorbent web material to form a pair of score lines that generally extend in the machine direction, the score lines defining the lateral flaps.

33. A method as defined in claim 22, wherein the absorbent structure has a length and wherein the lateral flaps extend substantially the entire length of the absorbent structure.

34. A method as defined in claim 22, wherein each individual absorbent pad has a rectangular shape prior to folding the lateral flaps.

35. A method as defined in claim 22, wherein by folding the lateral flaps, the lateral flaps create a basis weight in the location of the middle portion that is at least about twice the basis weight of areas of the front portion.

36. A method as defined in claim 32, further comprising the step of debulking the strip of absorbent web material.

37. A method as defined in claim 22, wherein the strip of absorbent web material has a basis weight of from about 100 gsm to about 2000 gsm.

38. A method as defined in claim 22, wherein by folding the lateral flaps, the lateral flaps create a basis weight in the location of the front portion of each absorbent pad that is at least about twice the basis weight of areas of the rear portion.

39. A method as defined in claim 22, wherein by folding the lateral flaps, the lateral flaps create a basis weight in the location of the middle portion that is at least about 25% greater than the basis weight of areas of the rear portion.

40. A method as defined in claim 22, wherein by folding the lateral flaps, the lateral flaps create a basis weight in the location of the middle portion that is at least about 150% greater than the basis weight of areas of the rear portion.

41. A method as defined in claim 22, wherein the absorbent web material contains synthetic binder fibers.

42. A method as defined in claim 22, wherein the absorbent web material contains an adhesive.

43. An absorbent article comprising:
an outer cover material;
a liner; and
an absorbent structure positioned between the outer cover material and the liner, the absorbent structure including a front portion, a rear portion, and a middle portion, the absorbent structure having a length and a pair of opposing lateral flaps that extend substantially the entire length of the absorbent structure, the pair of opposing lateral flaps being folded onto the front portion, the rear portion and the middle portion, the middle portion having a narrower width than the front portion and the rear portion, and wherein, once the lateral flaps are folded, the lateral flaps create at least a two layer structure in the location of the middle portion.

44. An absorbent article as defined in claim 43, wherein the absorbent structure further comprises a pair of opposing middle openings spaced between the middle portion and the lateral flaps, the middle portion being narrower than the front portion due to the location of the opposing middle openings.

45. An absorbent article as defined in claim 44, wherein the absorbent structure further comprises at least one rear opening.

46. An absorbent article as defined in claim 44, wherein the absorbent structure further comprises two opposing rear openings separated by a strip of material, the strip of material being narrower than the middle portion.

47. An absorbent article as defined in claim 46, wherein each of the rear openings is connected to a corresponding middle opening.

48. An absorbent article as defined in claim 43, wherein the absorbent structure has a rectangular shape when placed in an unfolded state.

49. An absorbent article as defined in claim 43, wherein the absorbent structure includes score lines that separate and define the lateral flaps.

50. An absorbent article as defined in claim 43, wherein the middle portion has a basis weight that is at least about twice the basis weight of areas of the rear portion.

51. An absorbent article as defined in claim 43, wherein the absorbent article is one of diapers, child's training pants, feminine care articles, and incontinence articles.

52. An absorbent article as defined in claim 43, wherein the absorbent structure comprises pulp fibers and superabsorbent particles.

53. An absorbent article as defined in claim 43, wherein the absorbent structure comprises an air formed web.

54. An absorbent article as defined in claim 43, wherein the basis weight of the absorbent structure is from about 100 gsm to about 2000 gsm.

55. An absorbent article as defined in claim 43, wherein the absorbent structure has been debulked and has a density of from about 0.15 g/cc to about 0.4 g/cc.

56. An absorbent article as defined in claim 50, wherein the middle portion has a basis weight that is at least about twice the basis weight of areas of the front portion.

57. An absorbent article as defined in claim 43, wherein, once the lateral flaps are folded, the lateral flaps create a basis weight in the location of the middle portion that is at least about 25% greater than the basis weight of areas of the rear portion.

58. An absorbent article as defined in claim 43, wherein, once the lateral flaps are folded, the lateral flaps create a basis weight in the location of the middle portion that is at least about 150% greater than the basis weight of areas of the rear portion.

59. An absorbent article as defined in claim 43, wherein the absorbent structure contains synthetic binder fibers.

60. An absorbent article as defined in claim 43, wherein the absorbent structure contains an adhesive.
